(19) United States
(12) Patent Application Publication
(10) Pub. No.: US 2007/0225792 A1
(43) Pub. Date: Sep. 27, 2007

(54) Conformable vascular prosthesis delivery system

(76) Inventors: Juan Granada, Pearland, TX (US); Simon M. Furnish, New York, NY (US)

Correspondence Address: DIAMOND LAW OFFICE LLC, 1605 JOHN STREET, SUITE 102, FORT LEE, NJ, 07024, US

(21) Appl. No.: 11/726,987

(22) Filed: Mar. 24, 2007

Related U.S. Application Data
(60) Provisional application No. 60/785,577, filed on Mar. 24, 2006.

(51) Int. Cl. A61F 2/84

(52) U.S. Cl. 623/1.11; 623/1.12

(57) ABSTRACT

Novel approaches for a conformable vascular prosthesis delivery system are provided which overcome the limitations of existing high pressure balloons for delivering intravascular prostheses to the site of high-risk plaques. One embodiment involves a short balloon segment which is inflated at one end of the prosthesis and then pulled to traverse the length of the prosthesis, dilating the surrounding prosthesis and securing it to the vessel wall as it traverses the length of the prosthesis. The short balloon segment causes less local trauma to the vessel relative to a full length balloon. Another embodiment involves use of a self-expandable mesh to expand the surrounding prosthesis and secure it to the vessel wall. The self expandable mesh is less traumatic than a typical angioplasty balloon because of the lower radial forces applied and the relatively higher transverse flexibility of the mesh.

This application claims the benefit of U.S. provisional patent application Ser. No. 60/785,577 filed Mar. 24, 2006, which is incorporated by reference herein in its entirety.

### FIELD OF THE INVENTION

The invention relates generally to the field of catheter-based delivery systems for endoluminal vascular prostheses.

### BACKGROUND OF INVENTION

Vascular stents are commonly used today for percutaneous transluminal angioplasty (PTA) that involve the delivery and deployment of a self expandable or balloon expandable stent to create a scaffolding for both improving and maintaining patency in diseased or otherwise constricted vessels.

Self-Expanding (SE) stents are typically constructed from Stainless Steel or Nitinol, either from laser cut and electro-polished tubing or welded wire braids, coils or other wire mesh forms that allow for a small unexpanded profile to reach distal lesions in tortuous vessels which can be deployed and expanded in place when released from a captive sheath. SE stents are less common in coronary applications and typically require both pre and post dilatation with an angioplasty balloon. Not only does this require the use of two or more device interventions to achieve the desired outcome, but the nature of the self expanding stent allows for continued long-term expansion in the vessel even 7 to 9 months after implantation, resulting in increased vessel injury. The advantages and disadvantages of SE coronary stents is still debated by physicians, but the global market shows that balloon expandable stents are in widespread use and considered the standard in PTA treatment.

Balloon expandable stents are plastically deformed via high pressure semi-compliant balloons and sized for a particular vessel. The balloon expandable coronary stents do not continue to expand after implantation and in some cases require no pre-dilatation. While typical balloon angioplasty, with or without a stent has shown definite acute improvements to the state of treatment of heart disease, but less of an effect on long term outcomes and survival. Angioplasty is a very traumatic process, primarily due to the high strains induced in the vessel wall from both radial expansion and straightening of a curved vessel. Stents are now being treated with drugs, radioactive seeds, thermal and cryogenic temperatures to counter the problem of restenosis, where the natural reaction to the implant causes proliferation of neointimal growth that may further reduce the diameter of a vessel. These provisions are essentially attempts to patch the damage incurred by the original treatment in order to provide a true long term benefit to the patient.

A new approach to the treatment of diseased vessels is recommended to reinvestigate the foundations of a minimally invasive approach to treating heart disease. While angioplasty is far less invasive when compared to coronary bypass surgery, there is a constant push to find further techniques to limit the damage caused by the basic procedure in order to treat a disease. One such approach involves the use of low radial force (lower than that of conventional stents), conformable endoluminal vascular prostheses to promote the formation of a normal intima at the treatment site.

In addition to atherosclerotic lesions requiring angioplasty or removal/ablation of occlusions generally, vulnerable plaques, which are sometimes known as high-risk atherosclerotic plaques, represent another indication for use of a low radial force, conformable endoluminal vascular prostheses that promote the formation of a normal intima. These vulnerable plaques include arterial atherosclerotic lesions characterized by a subluminal thrombotic lipid-rich pool of materials contained by and/or overlaid by a thin fibrous cap. Although vulnerable plaques are non-stenotic or nominally stenotic, it is believed that their rupture, resulting in the release of thrombotic contents, accounts for a significant fraction of adverse cardiac events.

In view of the above, there is a need for catheter-based delivery systems that are tailored for the delivery of low radial force, conformable endoluminal vascular prostheses.

### SUMMARY OF INVENTION

The present invention provides catheter-based delivery systems that are tailored for the delivery of low radial force, (lower than that of conventional stents used with angioplasty) conformable endoluminal vascular prostheses.

One embodiment involves a short balloon segment which is inflated at one end of the prosthesis and then pulled to traverse the length of the prosthesis, dilating the surrounding prosthesis and securing it to the vessel wall as it traverses the length of the prosthesis. The short balloon segment causes less local trauma to the vessel relative to a full length balloon.

Another embodiment involves use of a self-expandable mesh to expand the surrounding prosthesis and secure it to the vessel wall. The self expandable mesh is less traumatic than a typical angioplasty balloon because of the lower radial forces applied and the relatively higher transverse flexibility of the mesh.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIGS. 1-3 show various aspects of a direct balloon pullback delivery system embodiment of the invention.

FIGS. 4-5 show various aspects of a balloon-in-a-balloon pullback delivery system embodiment of the invention.

FIGS. 6-16 show various aspects of a captive prosthesis with balloon pullback delivery system embodiment of the invention.

FIGS. 17-23 show various aspects of a captive prosthesis with balloon push delivery system embodiment of the invention.

FIG. 24 shows an expandable mesh-based prosthesis delivery system embodiment of the invention.

### DETAILED DESCRIPTION

The invention provides catheter-based delivery systems that are tailored for the delivery of low radial force, conformable endoluminal vascular prostheses, rather than the high radial force conventional stents that have typically been employed to treat stenotic arteries in conjunction with angioplasty. For example, low radial force prostheses may include those exerting a radial force in the range of 30-250 mm Hg.

One embodiment of the invention provide a balloon-based delivery system that employs a short balloon segment to initiate expansion of a radially expandable, at least substantially tubular prosthesis from one fixed end, followed by the further radial dilation as the balloon is pulled, for example continuously without cycles of deflation and inflation, through the remaining length of the prosthesis. The shorter balloon is able to navigate more tortuous anatomy and can be inflated without forcing the vessel straight over the length of the balloon. The primary advantages offered by this embodiment are increased flexibility and decreased trauma as a result of reducing or eliminating the straightening effect.

Another embodiment of the invention provides a self-expanding mesh for the deployment, i.e. radial expansion, of an at least substantially tubular vascular prosthesis that surrounds the mesh. The flexible mesh is able to form around more tortuous anatomy without forcing the vessel straight over the length of the prosthesis. The primary advantages offered are increased flexibility and decreased radial trauma as a result of reducing or eliminating this straightening effect. This expandable mesh may be constructed in a similar manner as self expanding stents as described in the background—only in this case, the mesh is part of the delivery system and remains attached to the catheter once the prosthesis has been deployed. The mesh may require a coating, such as PTFE or Parylene to prevent adhesion to the prosthesis.

Various further aspects and embodiments of the invention are described below with reference to the appended figures.

### EXAMPLE 1

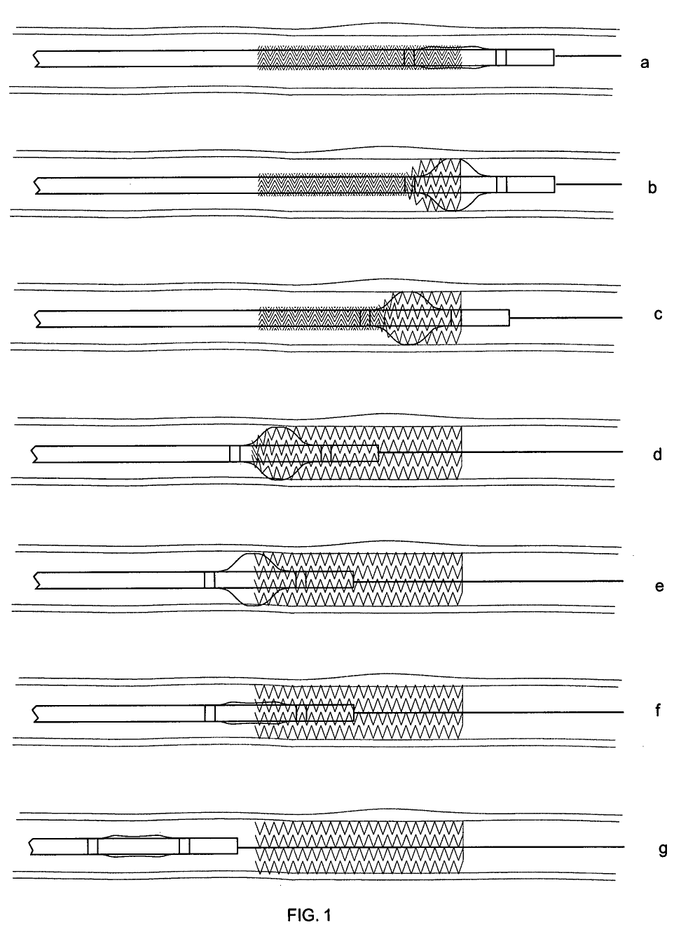

Example 1 illustrates a direct balloon pullback embodiment of the invention with reference to FIGS. 1-3.

A preferred embodiment includes a flexible catheter shaft similar to a common PTCA balloon or Balloon Expandable Stent Delivery System. The shaft has both a guide wire lumen and an inflation lumen. The inflation lumen is in fluid connection with the inside of a small balloon near the distal end of the catheter, as in similar catheters commonly utilized in catheter labs. The balloon is collapsed or folded into a low profile segment for delivery. A vascular prosthesis or stent is loaded into position with its distal edge covering the central portion of the balloon segment, with the remaining length trailing off proximal to the balloon directly adjacent with the shaft. Radio-opaque marker bands may be provided at varying locations along the distal portion of the catheter shaft to allow the interventionalist to predict the initial and final expanded length of the prosthesis once delivered.

In this embodiment, the prosthesis or stent is uncovered. FIG. 1 shows a stent as a patterned mesh such as those commonly used in interventional procedures. The stent may be fabricated as a laser cut tube, wire braid, welded or brazed wire form pattern or other expandable structure. Typical materials for stents are 316L Stainless Steel, alloys of Niobium, Cobalt-Chromium and Molybdenum and Nitinol. In some cases, stents may be coated with therapeutic drugs/agents which may be embedded in a coating or directly onto the stent surface itself. The balloon must be located at the distal end of the stent so that upon inflation, the stent can be anchored into the vessel wall with sufficient support to allow for deployment of the rest of the stent upon pullback. The stent is secured to the balloon during this initial expansion step via a polymer bond, crimp, or heat set into the balloon. Once inflated, this security measure is defeated allowing the balloon to move independently of the stent for pullback and deployment of the rest of the stent. The sequence shown in FIGS. 1(a) through 1(j) illustrate inflation (b), pullback (c-e) and deflation (f) resulting in stent deployment.

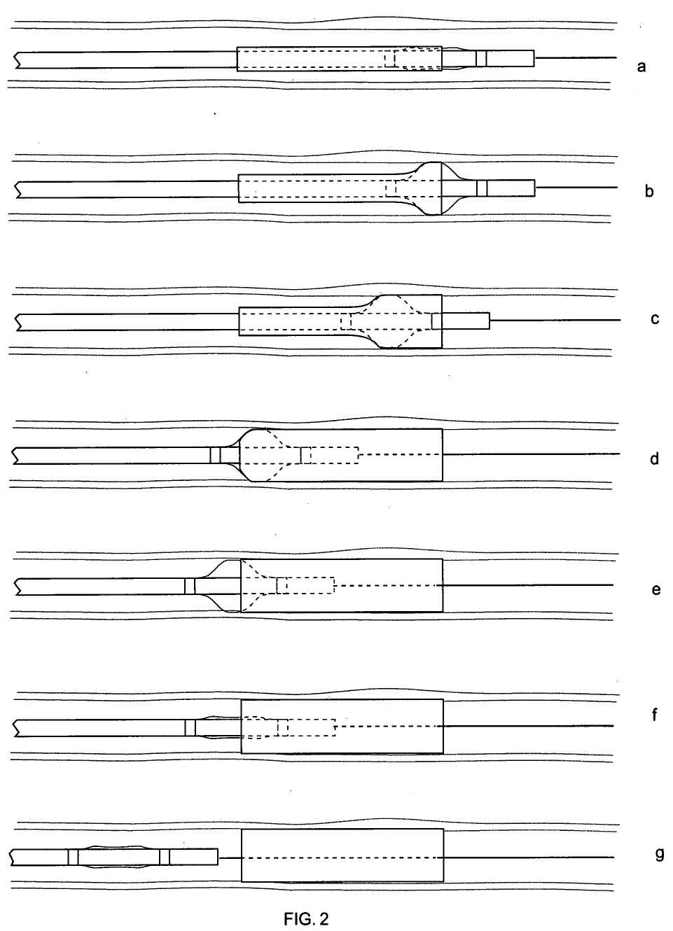

FIG. 2 shows a similar sequence for delivery of a thin-film luminal prosthesis. This embodiment is a slight variation on the delivery system shown in FIG. 1, but may be generalized to other vascular prostheses, including expandable tubular forms constructed from synthetic and natural materials that may be biodurable or biodegradeable/bioerodible.

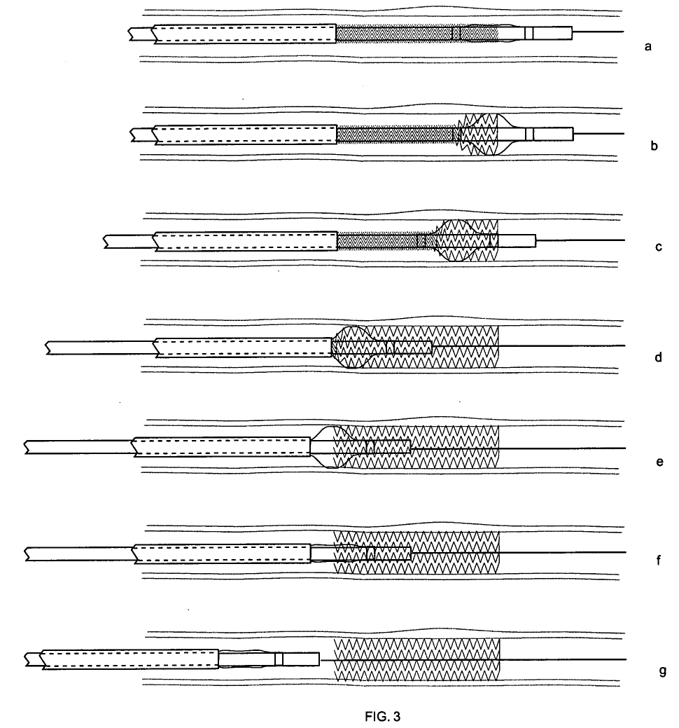

FIG. 3 shows an additional modification, with an outer sheath provided to help support the proximal end of the stent or prosthesis as the balloon is pulled through. Steps (a) through (d) show the balloon deployment and inner catheter shaft pulled to the left relative to the prosthesis and outer sheath. Step (e) in the sequence shows when the balloon is pulled up next to the outer sheath. The next step shows both the inner catheter and outer catheter pulled back in unison, deploying the final length of the stent or prosthesis prior to balloon deflation and removal.

The prosthesis may require additional anchoring to the vessel wall. One method of achieving this is to utilize an adhesive that is activated either by exposure to the surrounding fluids and tissues, via chemical catalyst or through exposure to an energy source, such as ultraviolet light. Transmission of chemicals and/or light can occur through extra lumens, optical fibers, etc. contained within the delivery system catheter or via a separate catheter or guidewire intended for this purpose. Examples of adhesives include cyanoacrylates, UV-cured cyanoacrylates, UV-cured acrylics, and protein linking compounds such as Naftalimide.

These embodiments can utilize compliant or semi-compliant balloons, depending upon the specific radial forces required to dilate both the prosthesis and vessel. Semi-compliant balloons expand to a nominal diameter under high pressures which can be increased slightly with increasing pressure. Semi-compliant balloons are particularly useful because of the predictability of the final inflated shape. In contrast, compliant balloons tend to expand in a manner that is far more dependent upon the surrounding environment. Once the “starting” inflation pressure is reached, the expansion advances sharply with increasing pressure. A latex balloon is an example of a compliant balloon. A mylar balloon, for example, can be formed into a far greater variety of shapes and are typical of a semi-compliant balloon. Typically, compliant balloons are constructed from elastomeric materials such as silicone, latex rubber and polyurethanes. Noncompliant balloons are typically constructed from polyamides (e.g., nylon), polyesters (e.g., mylar) and other high strength thermoplastics and thermosets.

### EXAMPLE 2

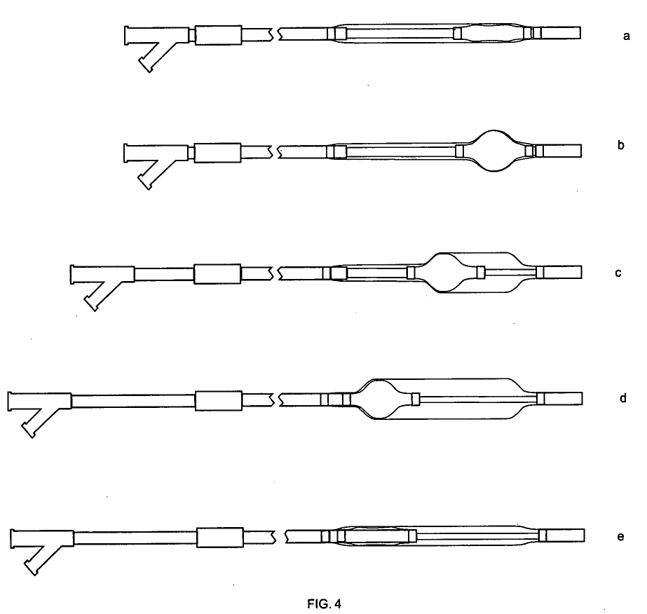

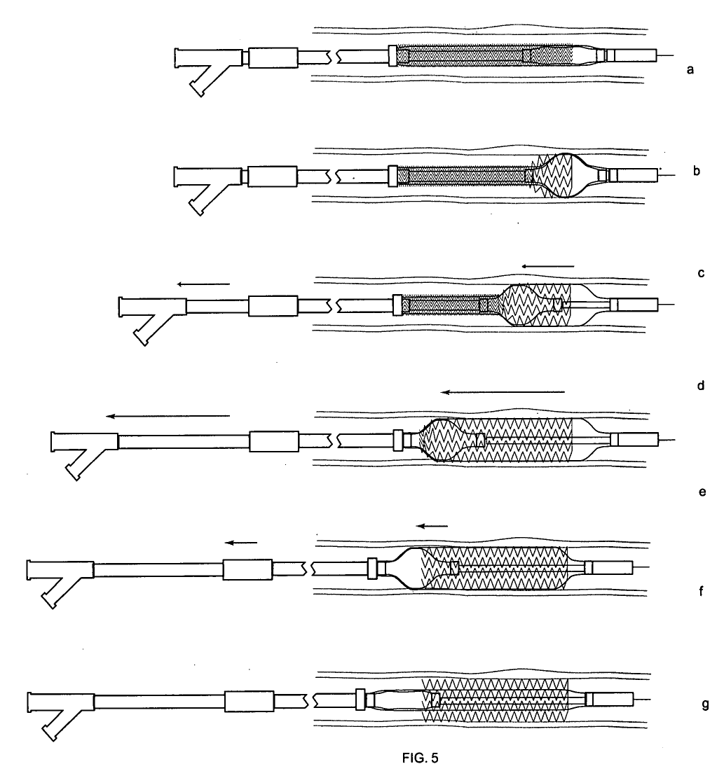

Example 2 illustrates a balloon-in-a-balloon pullback embodiment of the invention with reference to FIGS. 4-5.

This example illustrates an alternative embodiment to that of Example 1. Similar in function, this embodiment utilizes an expandable sleeve, which may be a secondary “balloon” which houses the smaller dilation balloon inside. This outer balloon is longer, residing beneath the full length of the prosthesis. FIG. 5 shows this configuration without the prosthesis in place. The outer balloon provides an expandable sleeve which permits facile sliding of the dilation balloon within it, but will not transmit the pull force from the dilation balloon to the prosthesis, thereby enabling a more controlled delivery and expansion. This outer balloon may be compliant or non-compliant. An alternate embodiment utilizes a secondary inflation lumen for filling this second balloon, for providing lubrication between the balloons and possibly to aid in collapsing the entire structure for removal. FIG. 5 shows the sequential operation of this “Balloon in a Balloon” delivery system with a patterned stent. This device may also be utilized for simple balloon dilatation of the vessel without a prosthesis.

### EXAMPLE 3

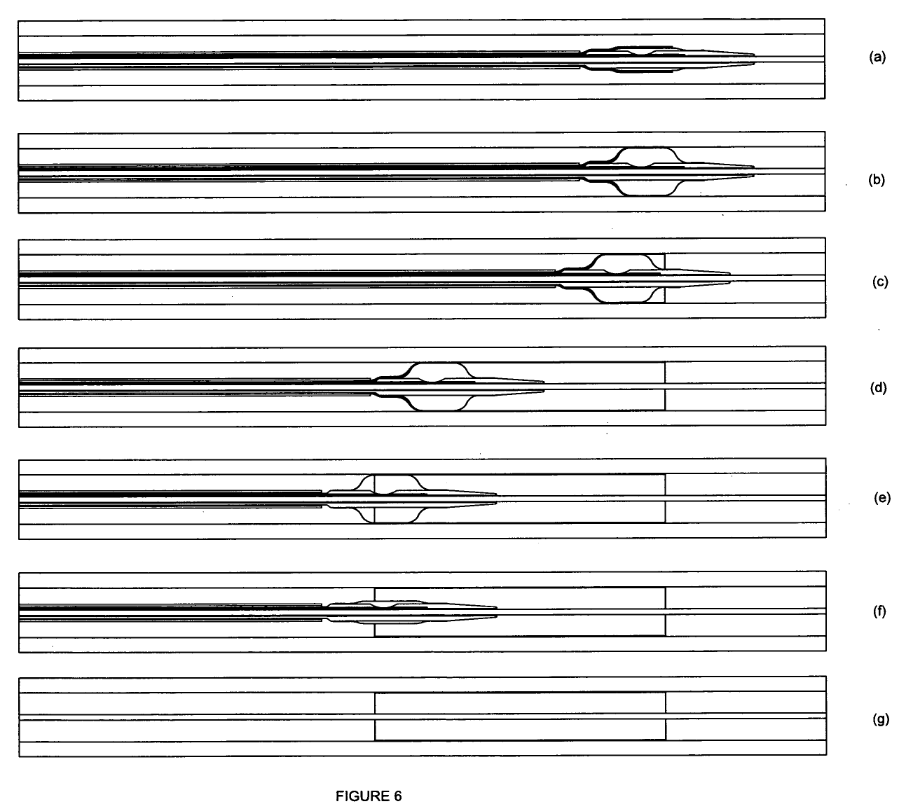

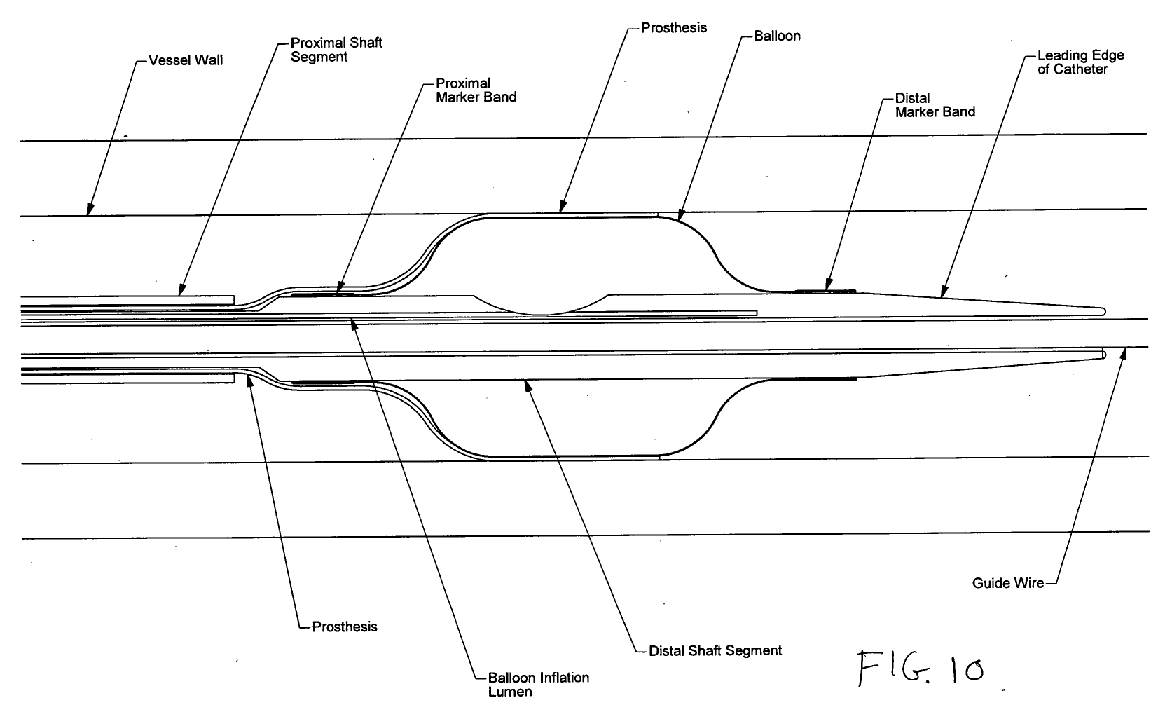

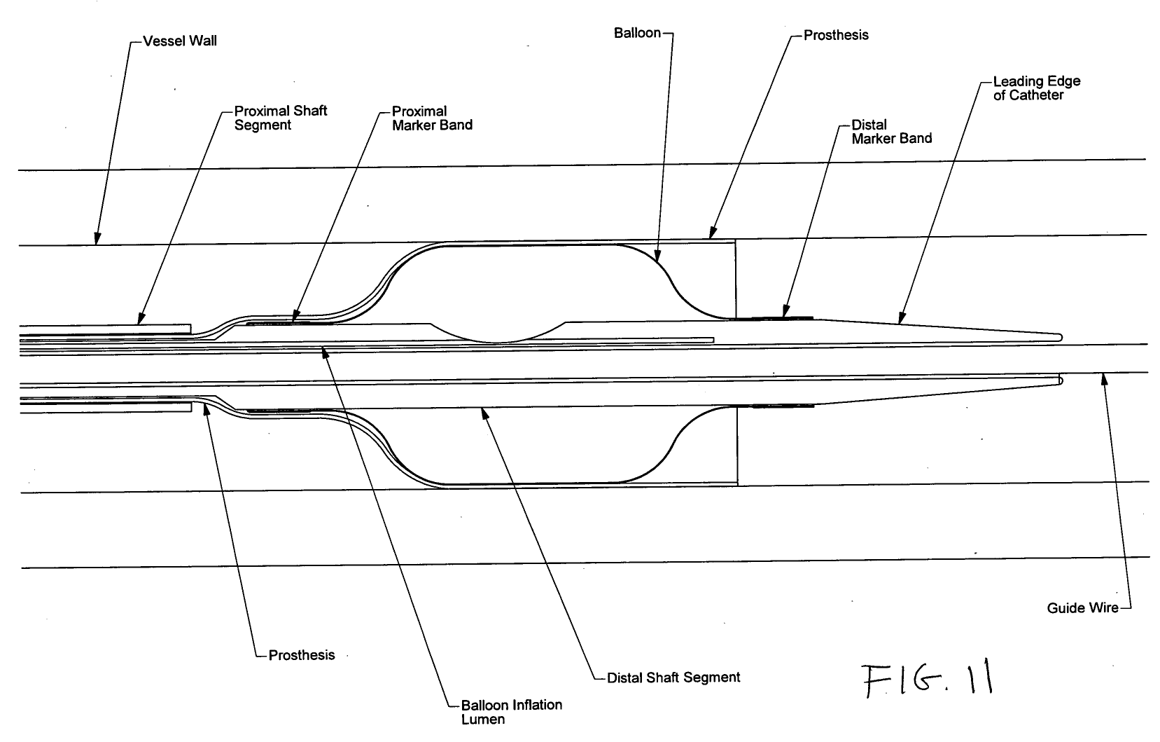

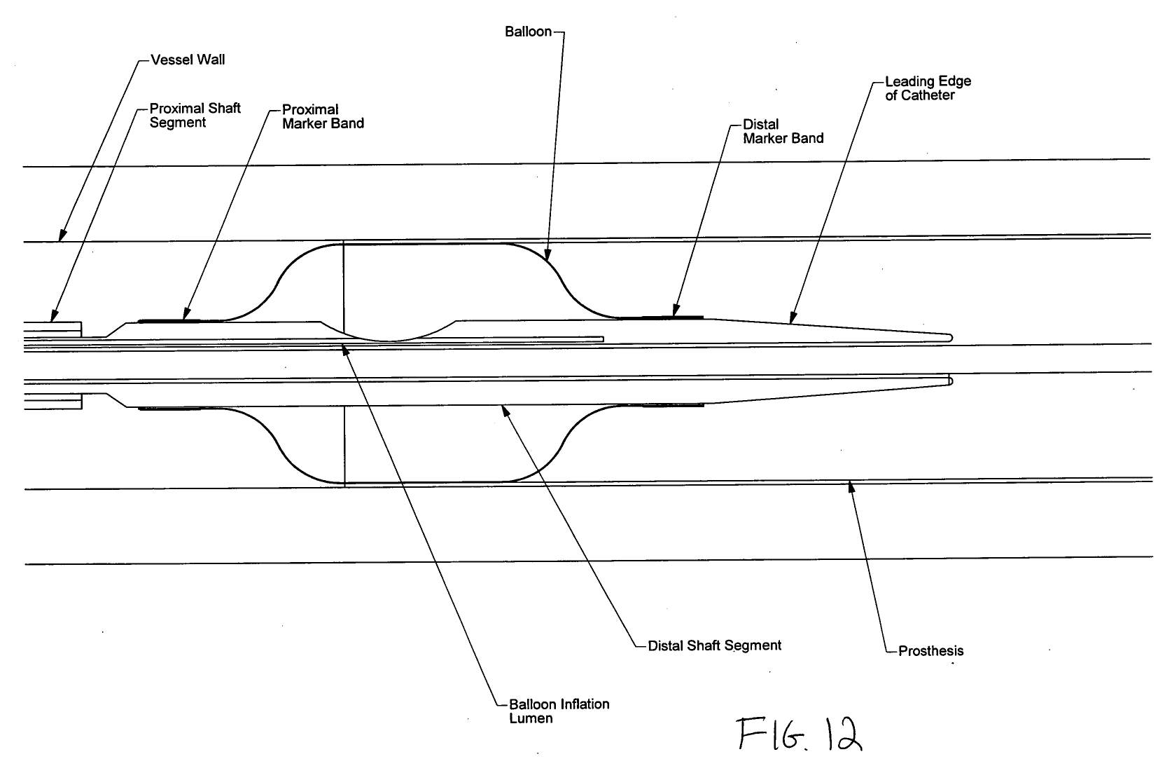

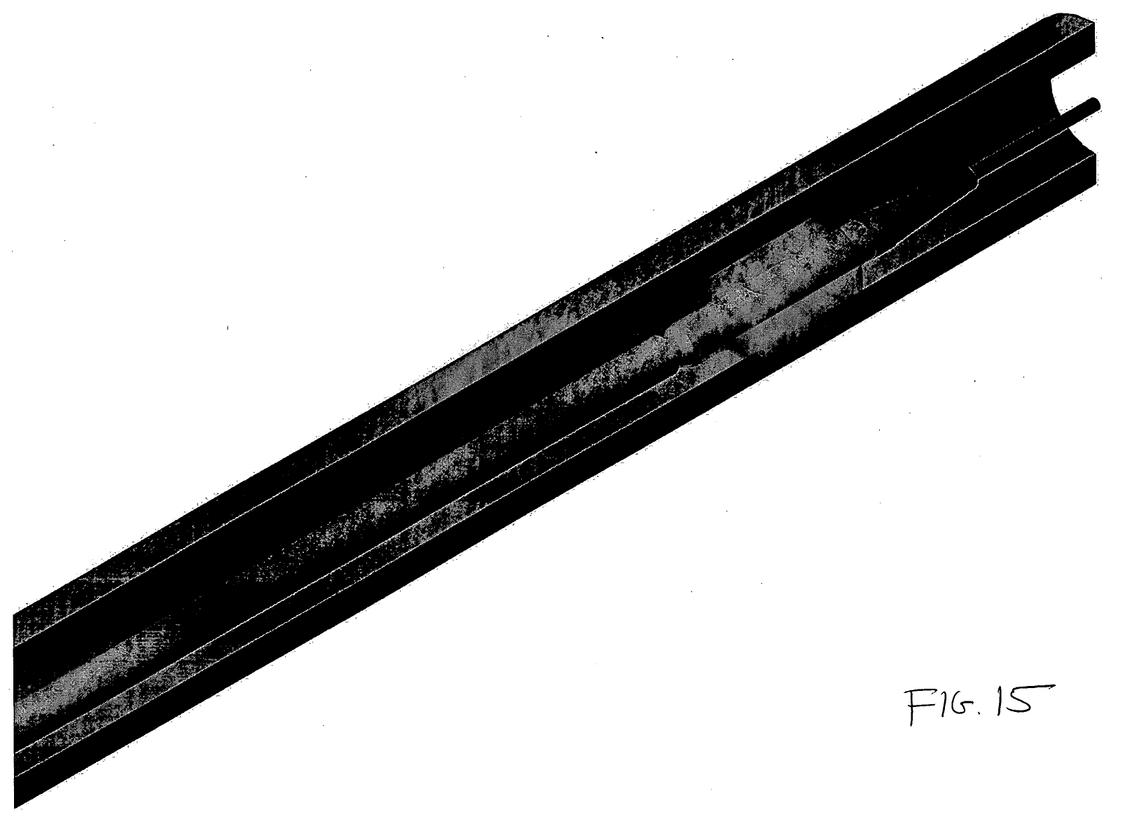

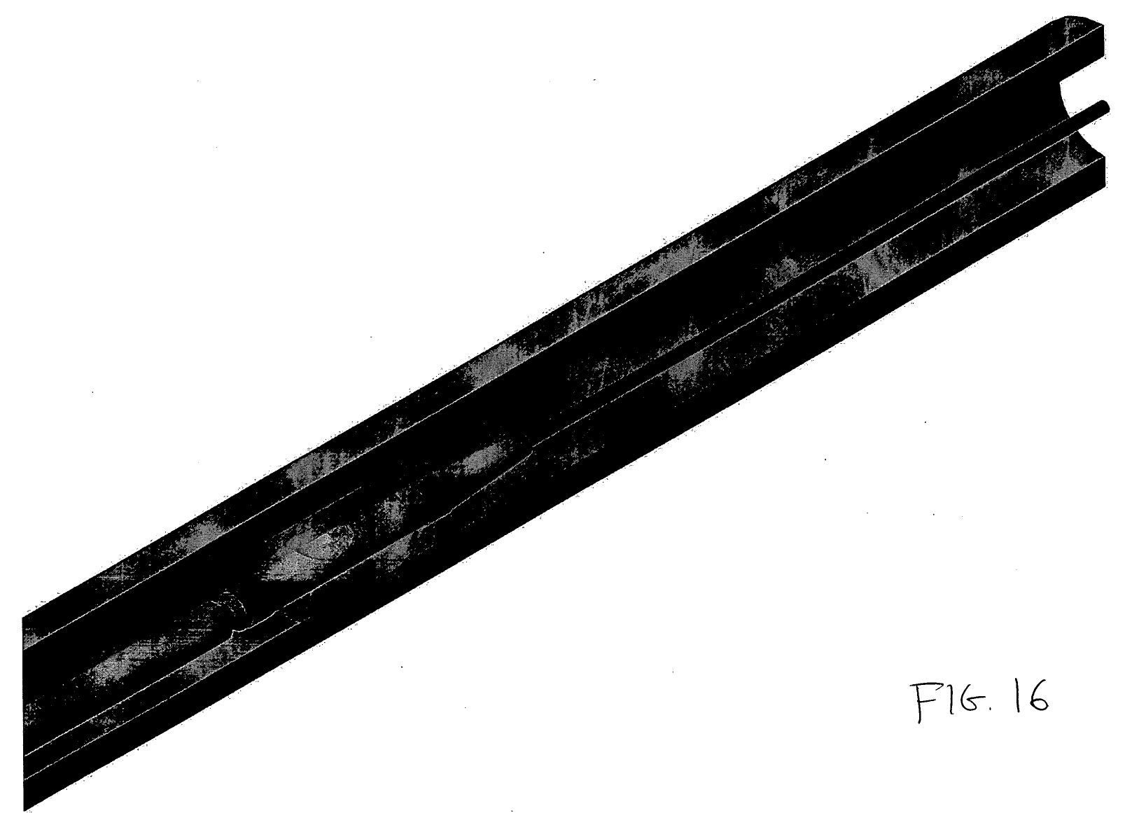

Example 3 illustrates a captive prosthesis with balloon pullback embodiment of the invention with reference to FIGS. 6-16.

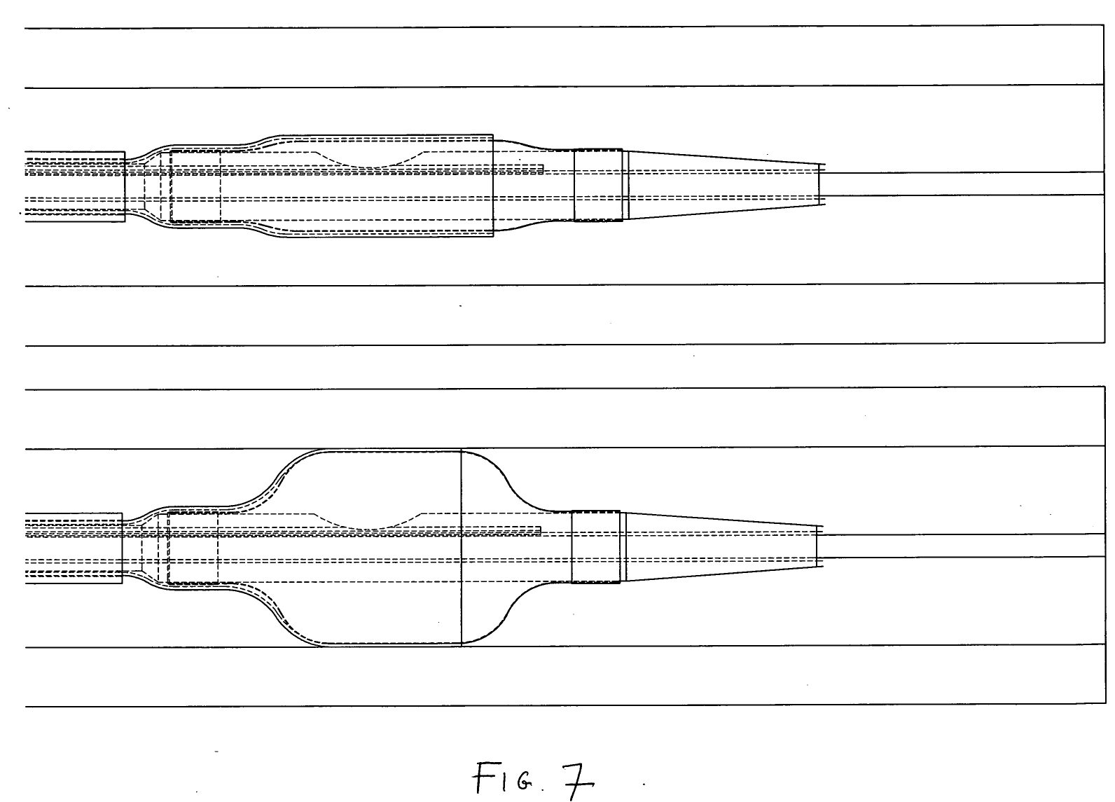

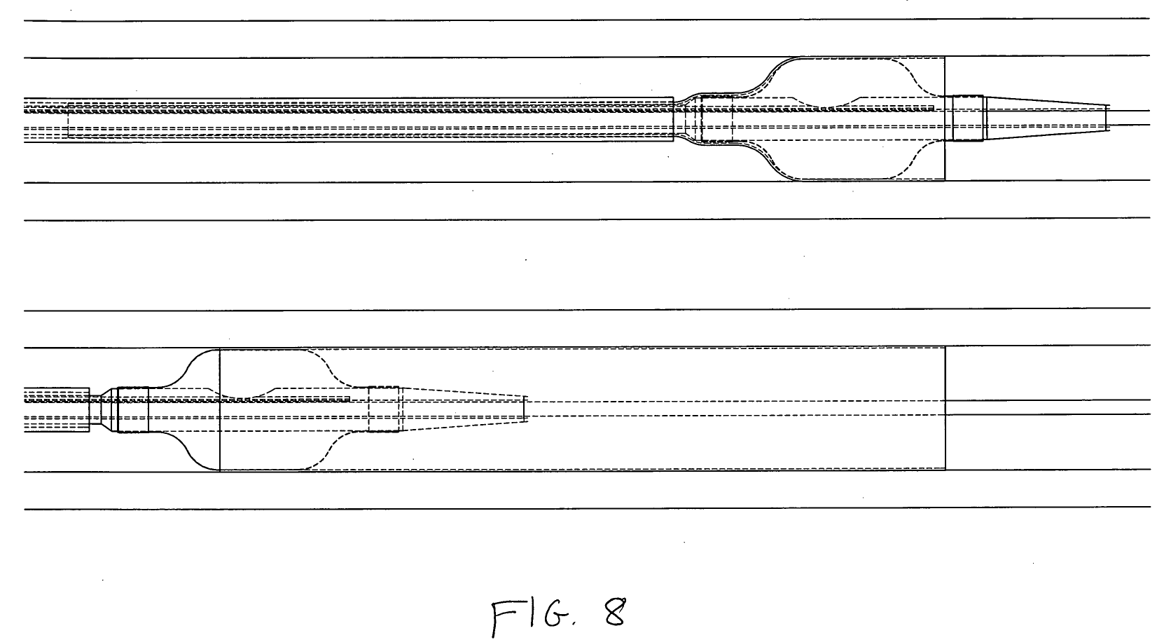

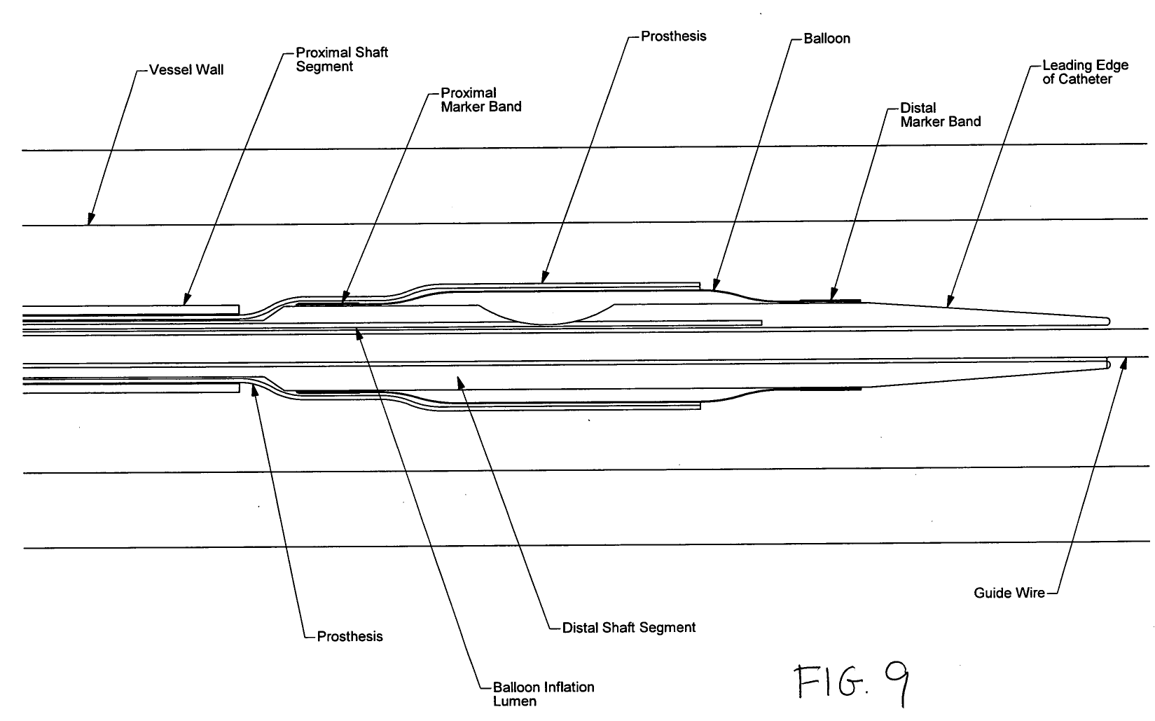

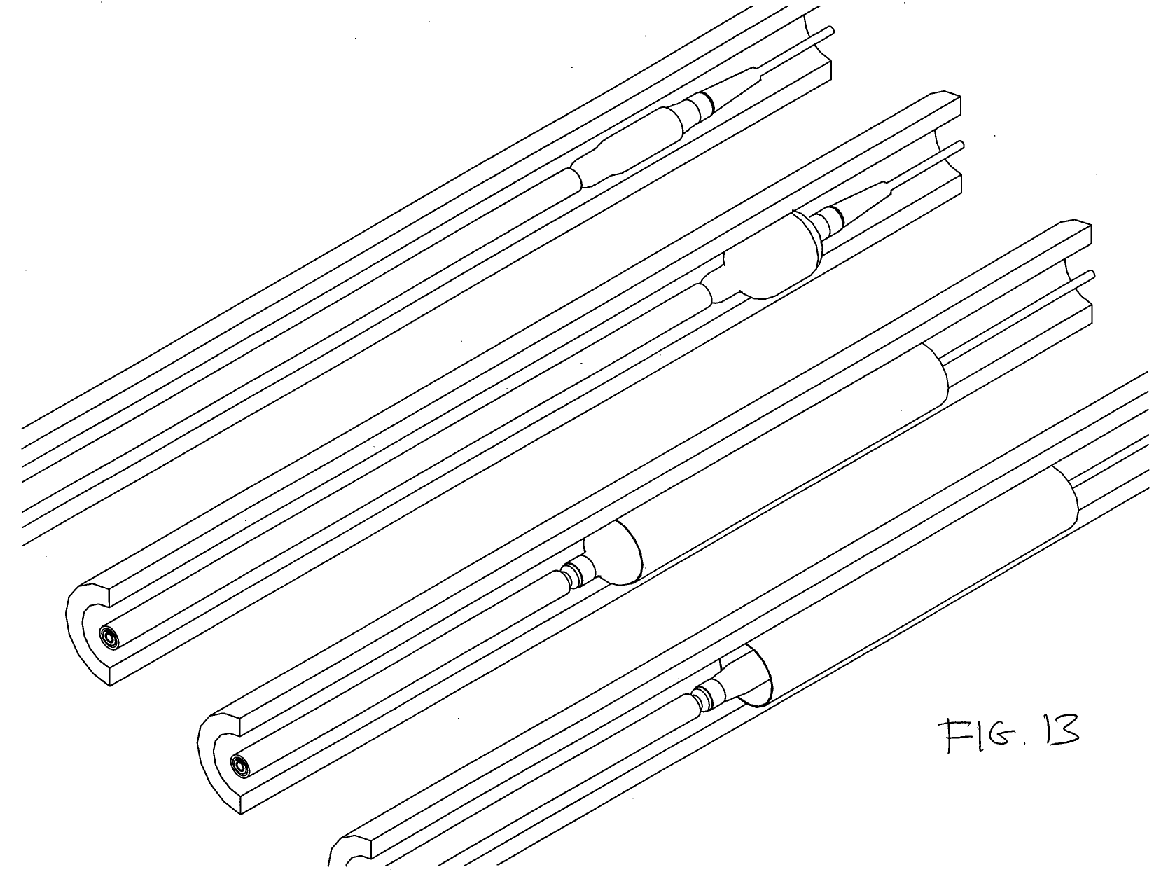

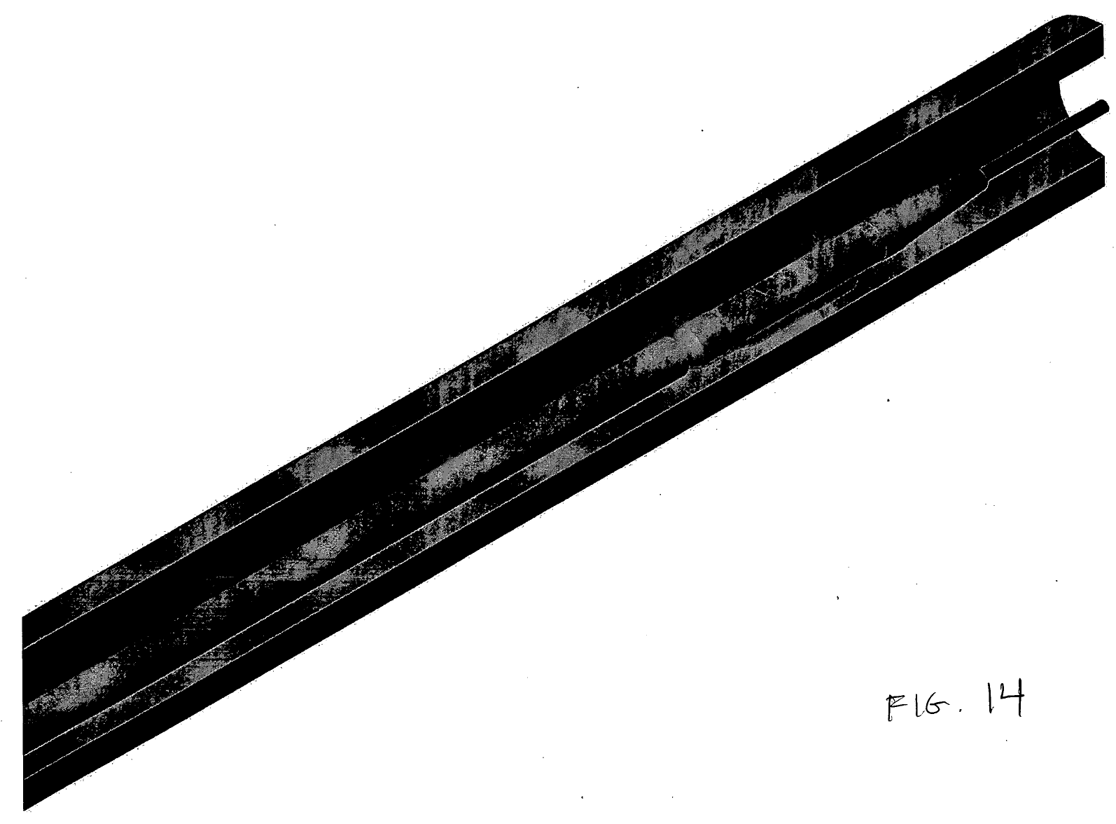

This alternate embodiment is similar to that of Example 1, with the addition of a thin sleeve over the prosthesis to protect it during delivery. As the balloon is expanded and drawn back, the flexible prosthesis is pulled from between the inner catheter shaft and outer sheath and expanded over the balloon into position at the vessel wall. FIGS. 6(a) thru (g) illustrate the sequential operation of this embodiment in section view. FIGS. 7 and 8 show an enlarged view to reveal the details of these same sequences. FIGS. 9-12 are detailed views with arrows indicating each component. FIG. 13 shows sequential isometric views of the prosthesis deployment within a sectioned vessel. FIGS. 14-16 show this same sequence with a full color representation and partially transparent balloon and prosthesis.

### EXAMPLE 4

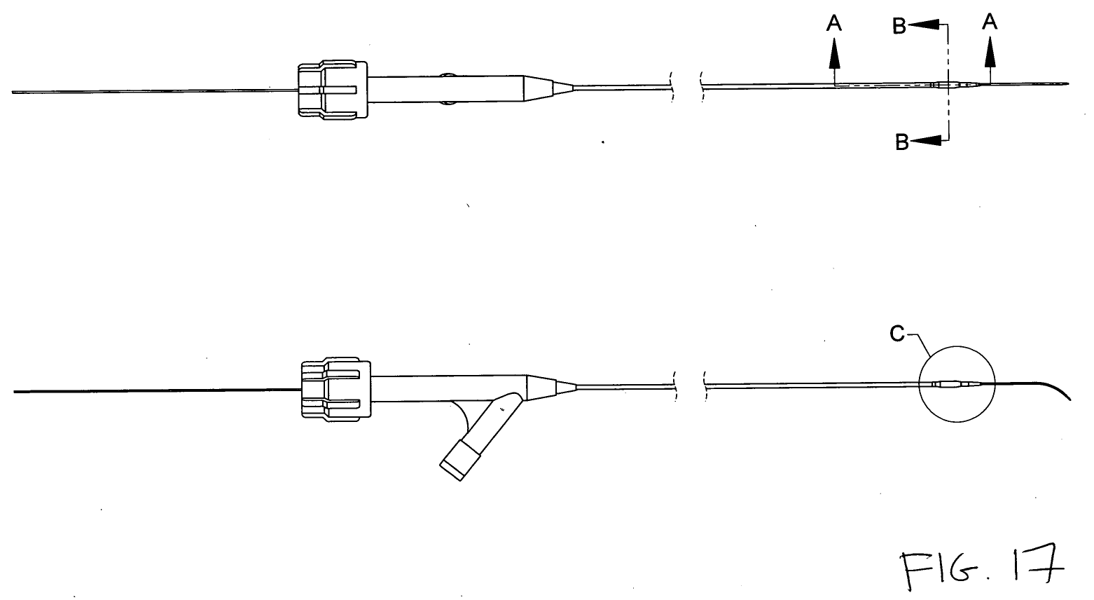

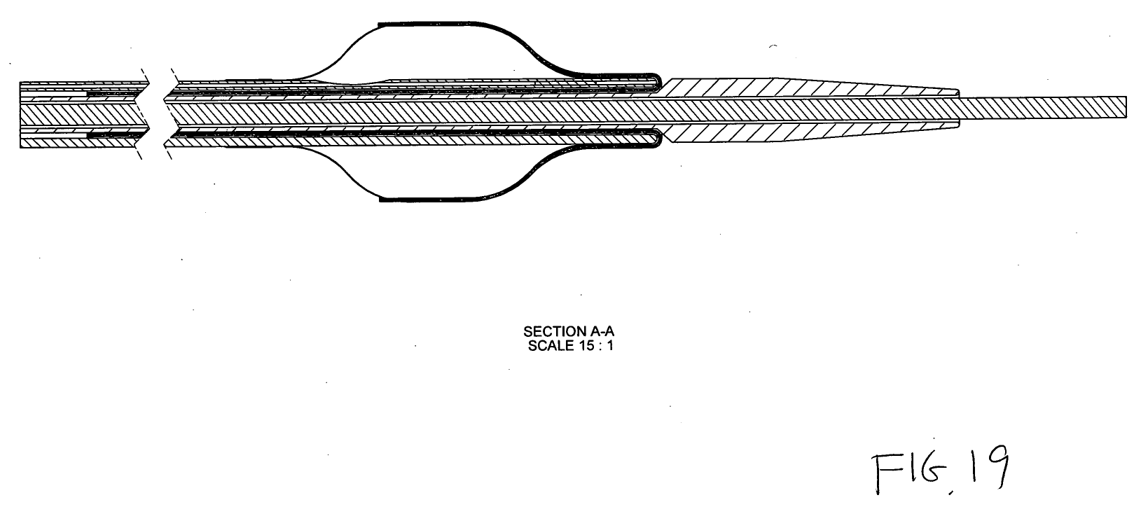

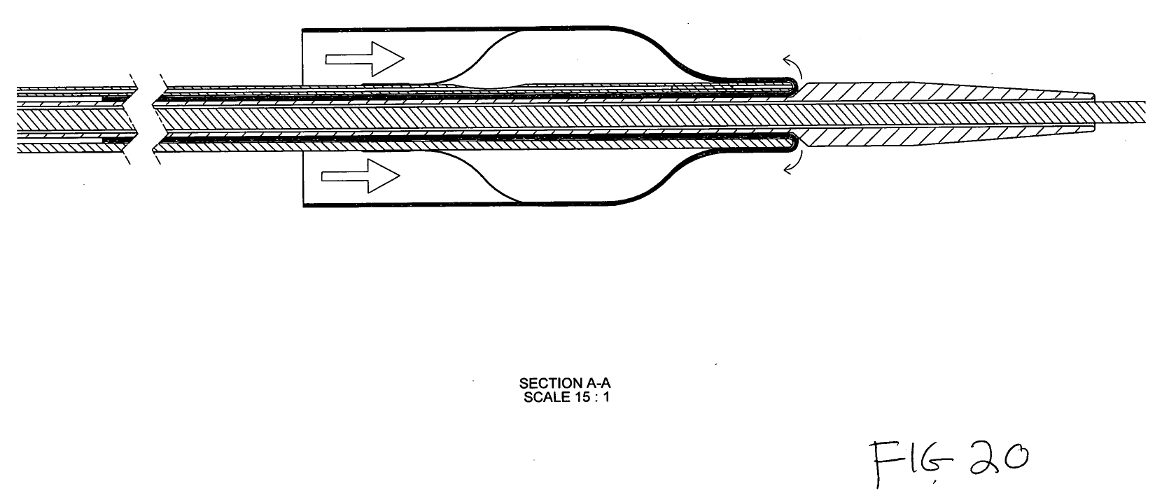

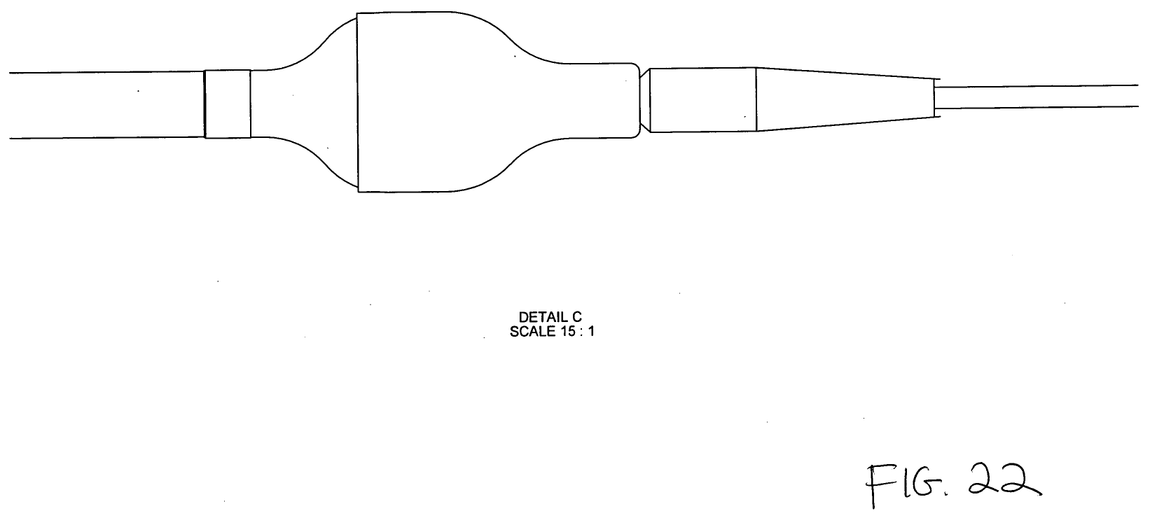

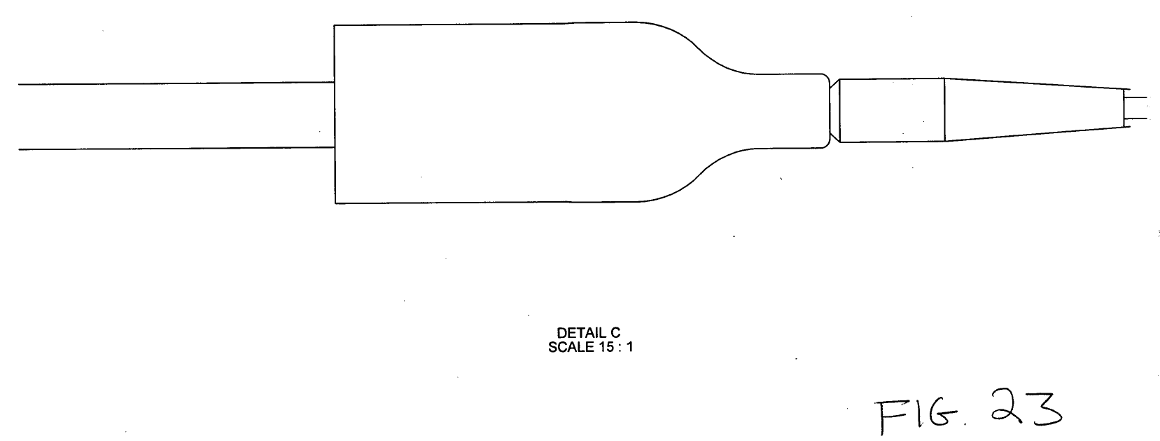

Example 4 illustrates a captive prosthesis with balloon push embodiment of the invention with reference to FIGS. 17-23.

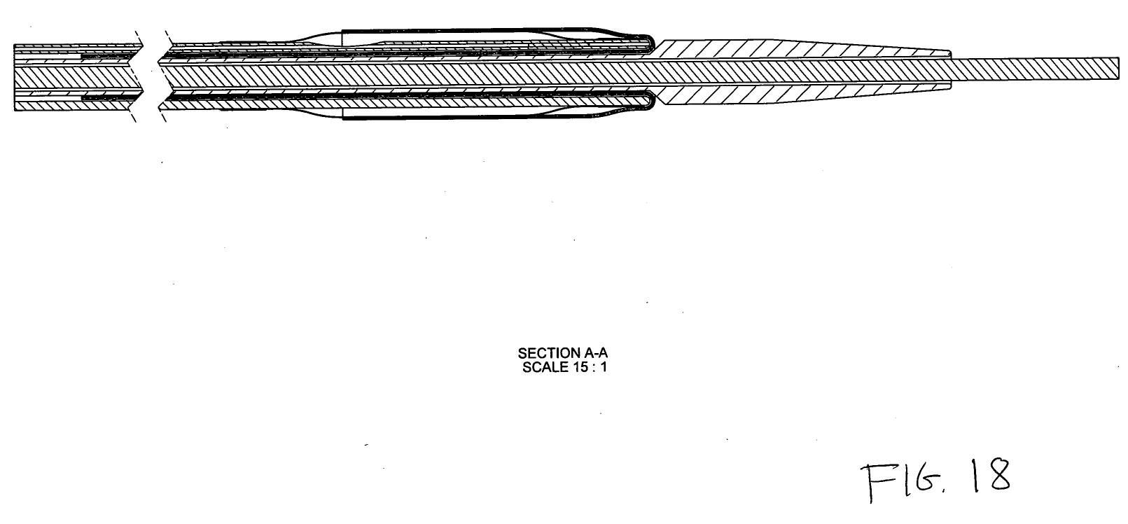

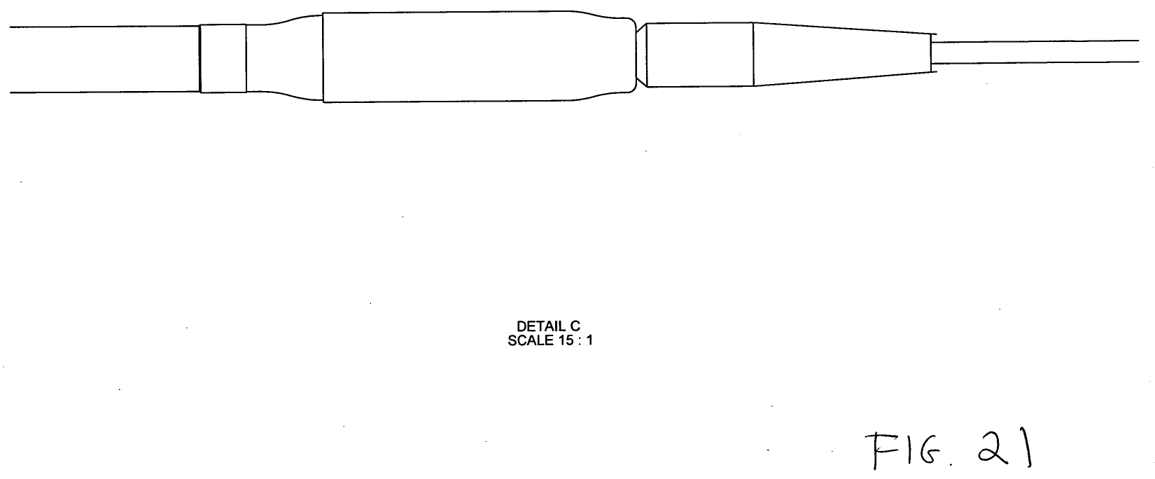

This alternate embodiment is similar to that of Example 3, but in a configuration for pushing the balloon forward for prosthesis deployment. In this embodiment, as the balloon is expanded and pushed forward the prosthesis is drawn out from the annular lumen between the primary shaft and the inner catheter shaft and inverted over the distal most termination of the outer tube and on to the short balloon segment. FIG. 17 shows a view of the catheter. FIGS. 18-20 illustrate the sequence of deployment for this embodiment, in section view indicated by Section A-A in FIG. 17. FIGS. 21-23 show a side view of the sequence from the detail “C” in FIG. 17.

### EXAMPLE 5

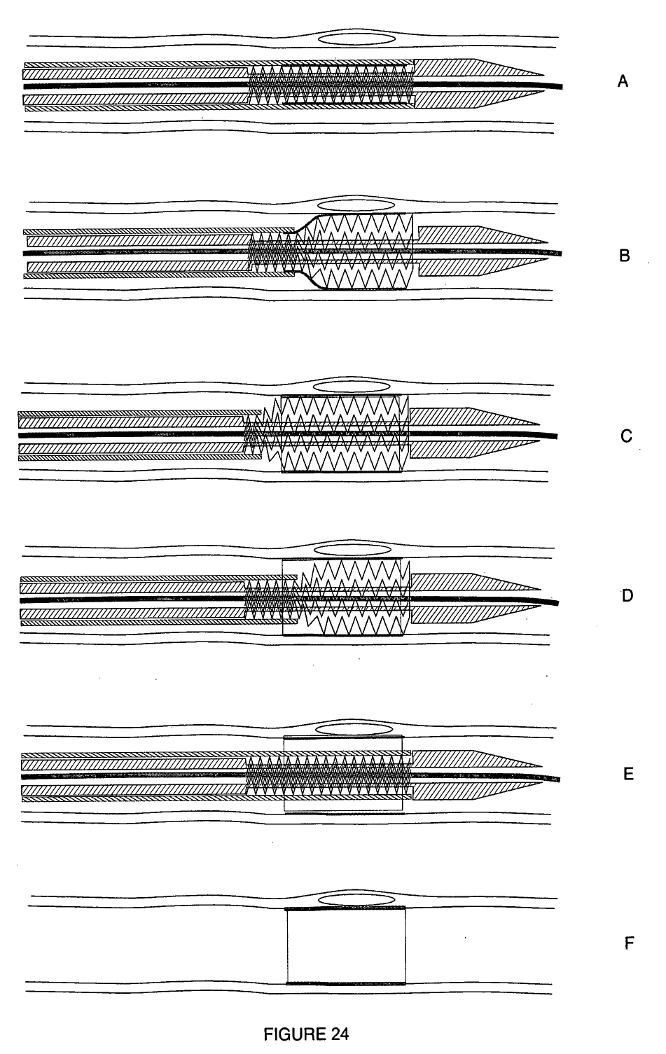

Example 5 illustrates an expandable mesh prosthesis delivery system embodiment of the invention with reference to FIG. 24.

This embodiment consists of a catheter containing an internal shaft and an external sleeve. The internal shaft contains a central guidewire lumen and a stepped cavity portion separating the proximal shaft portion from the distal tip portion. A self expandable mesh is attached to the proximal end of the cavity, compressed into a small diameter to fit between the internal shaft and outer sleeve. With the sleeve in its forward most position, the entire expandable mesh is forcibly compressed and held captive within the cavity. The proximal end of the mesh is fixed to the internal shaft. The prosthesis is wrapped or compressed onto the expandable mesh within the cavity. The delivery sequence is shown in FIG. 24.

FIG. 24(A) shows the catheter riding a central guidewire placed alongside a lesion. To deploy, the outer sleeve is pulled back through an external pullback handle manipulated by the physician as shown in (B). The outer sleeve is pulled back until the prosthesis is fully deployed (C). Then the sleeve is pushed forward relative to the inner shaft to recapture the mesh (D and E) and remove the catheter.

A membrane or cover (not shown) that surrounds the expandable mesh and permits the expansion thereof and is disposed between the expandable mesh and the prosthesis may also be provided to reduce friction between the expandable mesh and the prosthesis and to facilitate withdrawing the expandable mesh “back into” the catheter for removal of the catheter from the body. The membrane or cover may, for example, be a tube that connects to the catheter at or near the same position at which the expandable mesh is attached to the catheter.

### EXAMPLE 6

### Example 6 illustrates a Drug Delivery System embodiment of the invention.

This example is similar to the embodiment above, although rather than a prosthesis, the expandable mesh is coated with a drug, or therapeutic substance embedded in a thin film of material (e.g. microspheres, liposomes, lipids, biodegradable polymer, or hydrogel) which will adhere to the vessel wall upon contact. The mesh is expanded across the lesion for sufficient time to allow the drug to elute or adhere to the vessel wall, then it is recaptured and removed from the body. Possible drugs include antiproliferatives such as Paclitaxel, Sirolimus and Mitomycin C and their derivatives, or other therapeutic substances such as those currently utilized on drug eluting stents and balloon-based delivery drug delivery systems.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. A method for deploying a radially expandable intravascular prosthesis in a blood vessel, comprising the steps of:
providing a radially expandable intravascular prosthesis having an axial length, two opposite ends and a lumen;
providing an inflatable deployment balloon having an axial length smaller than the axial length of the prosthesis;
expanding the balloon within the lumen of the prosthesis at or near an end of the prosthesis to expand the prosthesis at or near the end; and
moving the expanded balloon toward the opposite end of the prosthesis to progressively expand the prosthesis along its axial length.

2. The method of claim 1, wherein the step of moving the expanded balloon toward the opposite end of the prosthesis to progressively expand the prosthesis along its axial length is performed without cycles of deflating and inflating the balloon.

3. The method of claim 1, wherein the step of moving the expanded balloon toward the opposite end of the prosthesis to progressively expand the prosthesis along its axial length is performed in a continuous motion without deflating the balloon.

4. The method of claim 1, wherein the axial length of the inflatable deployment balloon is no more than 30% of the axial length of the prosthesis.

5. A method for deploying a radially expandable intravascular prosthesis in a blood vessel, comprising the steps of:
providing an intravascular catheter having a proximal end and a distal insertion end and comprising at or near its distal insertion end:
a self-expanding deployment mesh attached to the catheter;
a radially expandable intravascular prosthesis surrounding the self-expanding mesh; and
a retractable retaining sheath enclosing and constraining the self-expanding deployment mesh and the radially expandable intravascular prosthesis;
inserting the catheter into a blood vessel; and
retracting the retractable retaining sheath to permit the self-expanding mesh to expand, thereby radially expanding the radially expandable intravascular to radially expand in the blood vessel.

6. The method of claim 5, further comprising the step of:
after the radially expandable intravascular radially expands in the blood vessel, withdrawing the self-expanding mesh into the catheter; and
withdrawing the catheter from the blood vessel.

7. The method of claim 5, wherein the step of after the radially expandable intravascular radially expands in the blood vessel withdrawing the self-expanding mesh into the catheter comprises moving the retractable retaining sheath back toward an unretracted position.

8. An intravascular catheter for deploying a radially expandable vascular prosthesis, comprising:
a proximal end and a distal insertion end and comprising at or near its distal insertion end:
a self-expanding deployment mesh attached to the catheter;
a radially expandable intravascular prosthesis surrounding the self-expanding mesh, said prosthesis being at least substantially reliant on expansion of the self-expanding deployment mesh for its radial expansion; and
a retractable retaining sheath enclosing and constraining the self-expanding deployment mesh and the radially expandable intravascular prosthesis.
